(19) United States
(12) Patent Application Publication
(10) Pub. No.: US 2007/0244430 A1
(43) Pub. Date: Oct. 18, 2007

(54) Multiple lumen epidural introducer

(76) Inventor: Raymond L. Bedell, Providence, UT (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/601,061

(22) Filed: Nov. 17, 2006

Related U.S. Application Data
(60) Provisional application No. 60/787,009, filed on Mar. 28, 2006.

(51) Int. Cl. A61M 31/00; A61M 5/178

(52) U.S. Cl. 604/93.01; 604/500; 604/164.13

(57) ABSTRACT

A multiple lumen epidural introducer includes at least two separate lumens each having a proximal port and a distal port; and a coupling interface for coupling the two lumens such that the two separate lumens can be inserted through a single point of entry.

### RELATED APPLICATION

The present application claims the priority under 35 U.S.C. §119 of previously-filed U.S. Provisional Patent Application No. 60/787,009, filed 28 Mar. 2006 and entitled, “Multiple Lumen Epidural Introducer Catheter/Sheath,” which provisional application is incorporated herein by reference in its entirety.

### BACKGROUND

The need to gain rapid, smooth and safe access to the epidural space of the spine is well documented. The epidural space is accessed in a number of medical procedures including, for example, spinal stimulation and the administration of anesthetics.

Typically, the epidural space is accessed using a needle that is inserted through the skin. The needle is followed by a guide wire along which a single lumen type epidural device can then be introduced to access the epidural space.

Sometimes, multiple attempts must be made using these tools to correctly access the epidural space. Multiple attempts to access the epidural space clearly causes increased trauma to the patient. Consequently, when the epidural space has been correctly accessed, it is highly desirable for the success of the procedure and the minimization of trauma to the patient that there be no need to change the entry site or remove the guide wire or needle until the procedure is completed.

However, some epidural procedures require a second entry site. For example, if a medical practitioner were placing a percutaneous spinal cord stimulator lead or performing lysis of lesions or scar tissue, multiple instruments are needed within the epidural space. Consequently, an additional entry site must be created or, in some procedures, the first instrument can be removed so that the second instrument can be inserted. Clearly, it can be inconvenient or problematic to remove a first instrument and insert a second. Alternatively, creating a second entry site using, for example, a second needle, guide wire, etc. causes additional trauma to the patient. Multiple entry sites, just like repeated attempts to gain proper access to the epidural space, significantly increase trauma, pain, and other risks including infection, bleeding, and nerve or Intrathecal injury.

### SUMMARY

A multiple lumen epidural introducer includes at least two separate lumens each having a proximal port and a distal port; and a coupling interface for coupling the two lumens such that the two separate lumens can be inserted through a single point of entry.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 is a schematic diagram of a multiple lumen epidural introducer, according to one exemplary embodiment.

FIG. 2 is a schematic diagram of a multiple lumen epidural introducer with guide wires, according to one exemplary embodiment.

FIGS. 3A and 3B are schematic diagrams of the distal ports of a multiple lumen epidural introducer, according to one exemplary embodiment.

FIGS. 4A and 4B are schematic diagrams of the distal ports of a multiple lumen epidural introducer, according to various exemplary embodiments.

FIG. 5 is a flowchart illustrating a method of inserting and placing a multiple lumen epidural introducer, according to one exemplary embodiment.

Throughout the drawings, identical reference numbers designate similar, but not necessarily identical, elements.

### DETAILED DESCRIPTION

A multiple lumen epidural introducer is described herein. The multiple lumen introducer allows multiple devices, such as needles, guide wires, stimulators, fluids and measuring devices, to simultaneously access the epidural space through a single point of entry into the body. The multiple lumen introducer can be inserted as accurately and easily as traditional single lumen epidural introducers.

Some previous medical devices have included multiple proximal and or distal openings for a common lumen. However, inserting multiple instruments through a common lumen can present problems in a procedure in the epidural space. For example, allowing the two leads of a spinal cord stimulator to contact can cause undesirable results. Other conventional introducers, such as vascular catheters, are too flexible to allow directed placement in the epidural space. Furthermore, most needles and introducers currently available are not suited for use with the wide variety of instruments and procedures conducted in the epidural space, such as spinal cord stimulators and fluoroscopy assisted placement.

However, while access to the epidural space is an ideal application for the device taught in this specification, the principles described herein may be applied to gain access to a variety of other tissues or body cavities.

As used herein and in the appended claims, the term “instrument” will be used broadly to refer to anything that might inserted through an introducer lumen during a medical procedure accessing the epidural space. Thus, the term “instrument” includes, but is not limited to, guide wires, measuring devices, imaging devices, electrodes, tubes, syringes, needles, catheters, fluids, anesthesia, etc.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

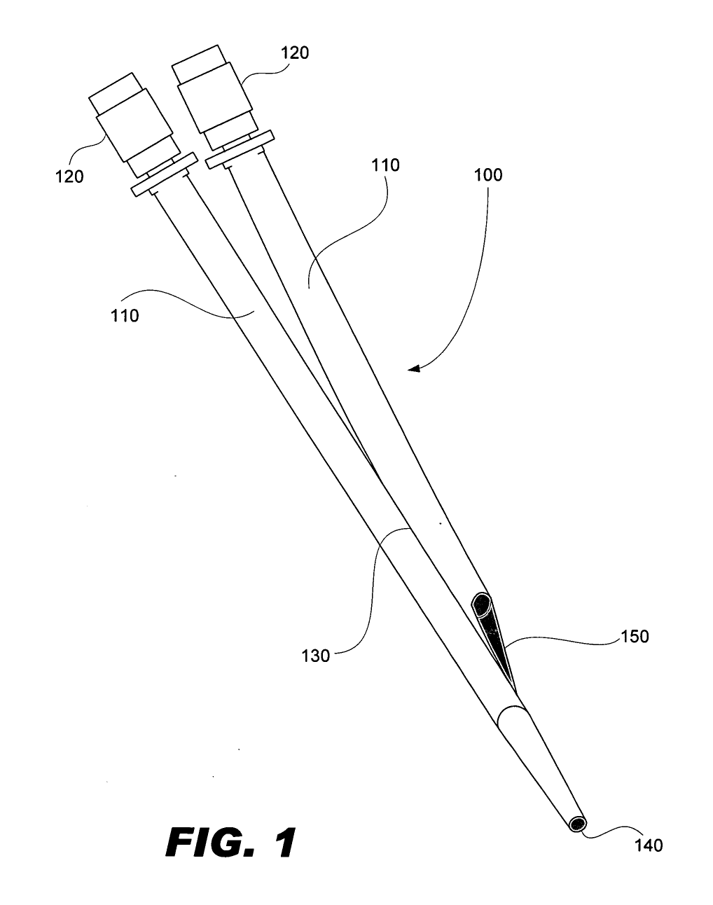

FIG. 1 is a schematic diagram of a multiple lumen epidural introducer, according to one exemplary embodiment of the principles described herein.

The multiple lumen epidural introducer (100) includes at least two lumens (110). While the lumens (110) are attached and cooperate to produce only a single point of entry through the patient's skin and subcutaneous tissue, there is no communication between the interiors of the lumens (110). Each lumen (110) represents a separate entry path for an instrument. Each lumen (110) includes an elongated tube forming a hollow sheath having at least one distal port (140, 150) and at least one proximal port (120). In the example of FIG. 1, the proximal ports (120) include luer locks. The lumens (110) may be substantially rigid to allow accurate placement within the epidural space and to maintain orientation and position after placement. According to one exemplary embodiment, the lumens (110) may be composed of plastic, although the principles described herein may also be practiced using lumens of metal or other synthetic or non-synthetic materials.

Fluoroscopic imaging may be used to facilitate the placement of the epidural introducer (100). The materials used in the epidural introducer may be radio-opaque so as to be visible using fluoroscopy. For example, the lumens and distal ports (140, 150) may be formed of plastic embedded with a metal or a compound containing barium, bismuth, or other radiation absorbing element. Radio-opacity enables the epidural introducer (100) to be viewed during fluoroscopy for accurate and safe placement.

As will be appreciated, the relative sizing of the lumens can be adapted as best suits a particular application. According to one exemplary embodiment, each lumen (110) may have an outer diameter greater than or equal to 8 french. According to another exemplary embodiment, each lumen (110) may have an outer diameter between 1 and 3 millimeters. Other embodiments may include lumens with larger or smaller diameters. Additionally, the two lumens may have different and/or varying diameters. Also, lumens with the same outer diameter are not required to have the same inner diameter.

As illustrated in FIG. 1, the two lumens may be of similar length. According to other embodiments, the lumens may be of substantially different lengths.

The lumens (110) are coupled so that the distal ends of the two lumens (110) are joined at and/or before the distal ports (140, 150). The proximity of the distal ports (140,150) allows for the introduction of multiple instruments through the two lumens (110) into the same area of the epidural space and minimizes the trauma to the patient of introducing the sheath (100).

In contrast, the proximal ends of the lumens (110) may be uncoupled to allow a variable separation distance between the proximal luer lock ports (120). This facilitates the insertion of two different instruments into the two separate lumens (110). At least the proximal portions of the lumens (110) may be made of a flexible material so that the two lock ports (120) can be moved further apart or closer together as best fits a particular application. Alternatively, the lumens (110) may be coupled over a substantial portion of the length of the lumens (110) for increased stability and precision, or the lumens (110) may be coupled over the entire length of the lumens (110), fixing the distance between luer lock ports (120).

As shown in FIG. 1, at least one coupling interface (130) is provided between the lumens (110). As used herein and in the appended claims, “coupling interface” is used broadly to mean a point at which the two lumens (110) are attached. As will be explained here, the lumens (110) may be formed as an integral unit, permanently coupled together or releasably coupled. In some examples, the lumens (110) may be glued, molded together, or otherwise attached at the coupling interface (130).

As indicated, in some examples, the lumens (110) are permanently coupled at the coupling interface (130). In other examples, the lumens (110) may be detached and reattached at the coupling interface (130). The replacement of a lumen may be useful, for example, if one of the attached lumens is damaged or if a different size or type of lumen is desired.

In some examples, the multiple lumen epidural introducer (100) may include multiple coupling interfaces for the attachment of more than two lumens (110). As illustrated in FIG. 1, the coupling interface may be located on the side of one or more lumens (110). Additional embodiments may include coupling interfaces (130) located at sites other than the side of a lumen (110). The coupling interface (130) is implemented so that the lumens (110) remain isolated, meaning that there is no communication between the interiors of the lumens (110).

As indicated, a variety of guide wires, instruments, devices, and fluids may be introduced into the body, particularly into the epidural space, through the lumens (110). Additionally, instruments, tubes, syringes, catheters, and other devices may be secured to the lumens (110) using the proximal ports (120) and may gain access to the body through the distal ports (140, 150).

According to one embodiment, the proximal ports (120) may be female luer locks. The luer locks may be a variety of sizes, and each proximal port (120) need not be the same size or shape. A variety of other methods or devices may be used to secure devices to the lumens (110), including, but not limited to male luer locks, threads, and other fasteners. In addition, attachments may be used to interface a proximal port (120) with another device, such as a tube that may not have the corresponding port for attachment.

According to the embodiment of FIG. 1, the opening of at least one distal port (140) is located at the distal end of a lumen (110). The opening of the distal port may be the same diameter as the associated lumen (110) or the opening may be larger or smaller than the diameter of the lumen (110). According to the embodiment of FIG. 1, the opening of a distal port (140) is substantially circular. Alternatively, the distal port (140) may have a variety of other shapes and may be angled so as not be perpendicular with, or along the same axis as, the shaft of the lumen (110). Additionally, multiple distal ports (140, 150) may be associated with a single lumen (110).

At least one additional lumen (110) includes a distal port (150). As illustrated, the distal port (150) may form a tapered opening. The taper of the opening allows the multiple lumen epidural introducer (100) to be inserted with minimal trauma to the patient at the entry site.

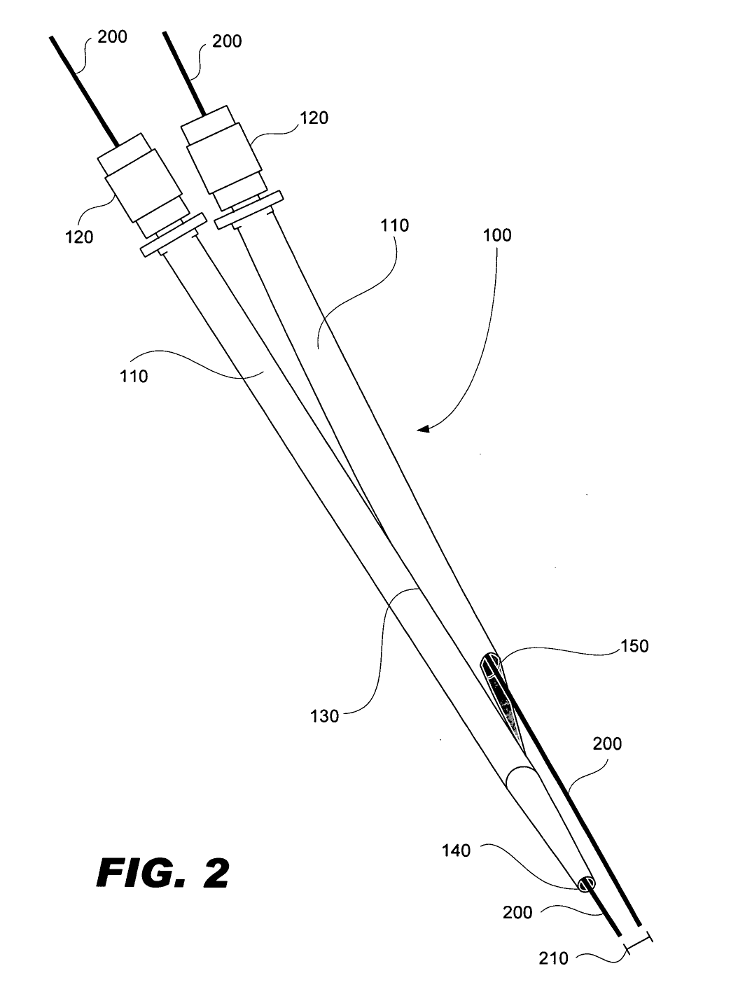

FIG. 2 is a schematic diagram of a multiple lumen epidural introducer being used with guide wires through the lumens, according to one exemplary embodiment.

As described herein, various instruments (200) including, but not limited to guide wires, measuring devices, fluids, and other instruments, may be inserted into the lumens of the multiple lumen epidural introducer (100) of FIG. 1. The instruments (200) inserted through the lumens (110) in the example shown in FIG. 2 are guide wires. The guide wires (200) are shown entering the proximal luer lock ports (120), extending through the lumens (110) and out the distal ports (140, 150). As described above, many other devices and instruments may be used with the multiple lumen epidural introducer (100). A number of support devices, including needles, catheters, Touhy needles, guide wires, syringes, loss of resistance (LOR) syringes, fluoroscopic imaging equipment, and other instruments may be used with the multiple lumen epidural introducer (100).

According to the exemplary embodiment of FIG. 2, each lumen (110) has one proximal port (120) and one distal port (140, 150). Additional embodiments may include lumens (110) with multiple proximal ports (120) and/or multiple distal ports (140, 150), and may have differing numbers of proximal ports (120) and/or distal ports (140, 150).

As illustrated, the proximal ports (120) may be separated by some distance, either fixed or adjustable distance, to facilitate manipulation of devices entering the lumens (110). It is advantageous for proximal ports to be separated so that a variety of instruments may be used simultaneously. For example, a separation between the proximal ports allows two syringes to be attached, even though the width of the syringes may be much larger than the width of the lumens (110). According to one embodiment, at least one portion of a lumen (110) may be somewhat flexible, providing a variable distance between the proximal ports (120) and/or lumens (110). A separation between the proximal ports facilitates, for example, the insertion or placement of an instrument (200) through a lumen (110) while maintaining the position or orientation of a previously placed instrument (200).

At the other end of the introducer (100), a separation distance (210) between the openings of the two or more distal openings (140, 150) facilitates the use of instruments that require separation or isolation. For example, the two leads of a spinal cord stimulator must not make contact in order to be effective. Isolated lumens (110) with a fixed separation between distal openings (140, 150) are ideal for certain procedures.

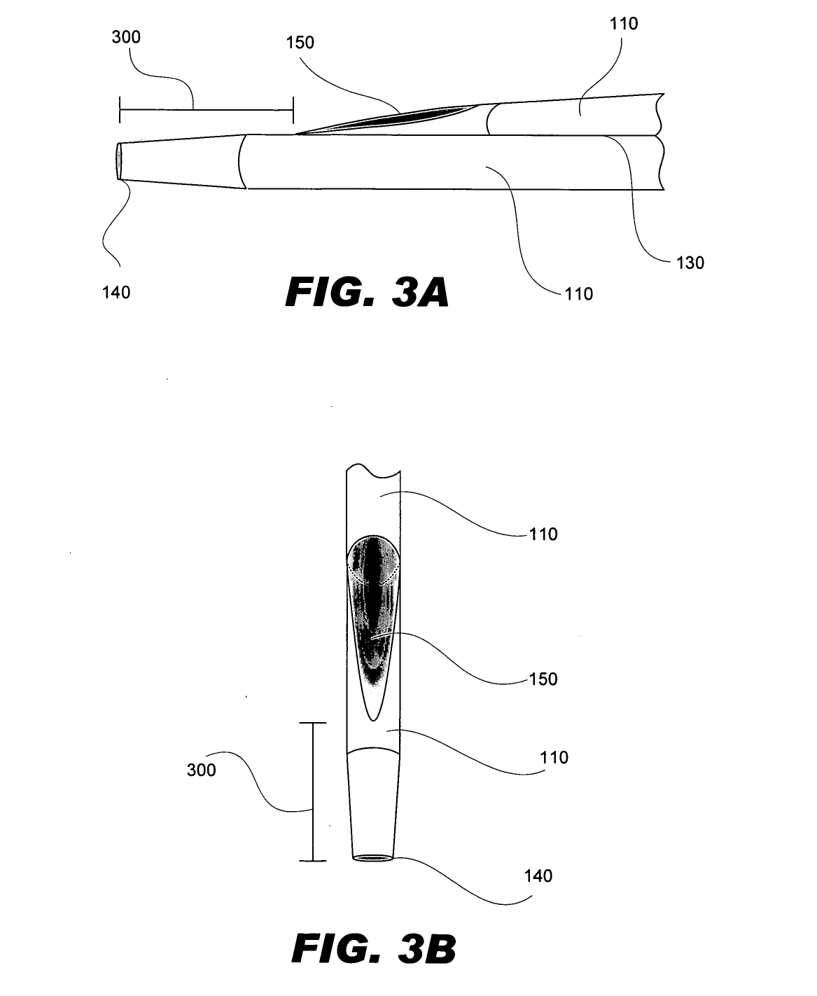

FIGS. 3A and 3B are schematic diagrams of the distal ports of a multiple lumen epidural introducer, according to one exemplary embodiment. FIG. 3A illustrates the side view of the distal ports and FIG. 3B illustrates an aerial view of the distal ports.

According to the embodiment of FIGS. 3A and 3B, a first distal port (140) is separated from a second tapered distal port (150) by a first distance (300) along the length of the epidural introducer (100). In some embodiments, it is advantageous for the separation distance (300) to be at least 20 millimeters. If the openings of the distal ports (140, 150) are closer than 20 millimeters to each other, two instruments inserted separately through the two lumens (110) may interfere with each other once inserted.

Additional embodiments, however, may include two or more distal ports (140, 150) that are not separated by a distance (300). For example, distal ports (140, 150) may be arranged close together to allow fluid to enter the body through one port while an instrument is inserted through another. Distal ports (140, 150) located close to each other may also be useful for certain procedures that require instruments, measurements and/or fluids to be inserted in close proximity.

Some embodiments may include three or more lumens and/or distal ports (140, 150) that may be spaced at various distances or may be arranged so that the openings of the distal ports (140, 150) are not all separated along the length of the lumens (120). One embodiment may include two openings at the distal tip of the epidural introducer (100), with a third distal port placed at least 20 mm away from the other distal openings.

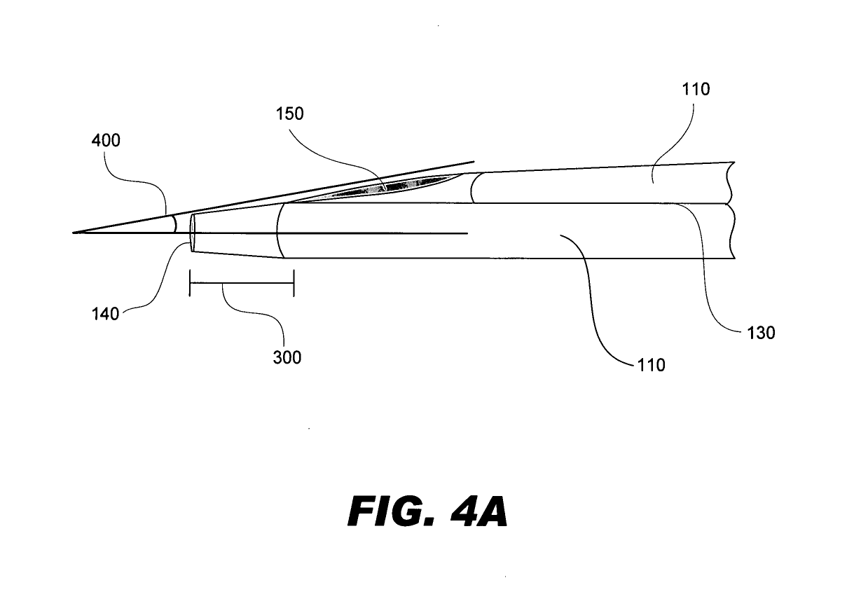

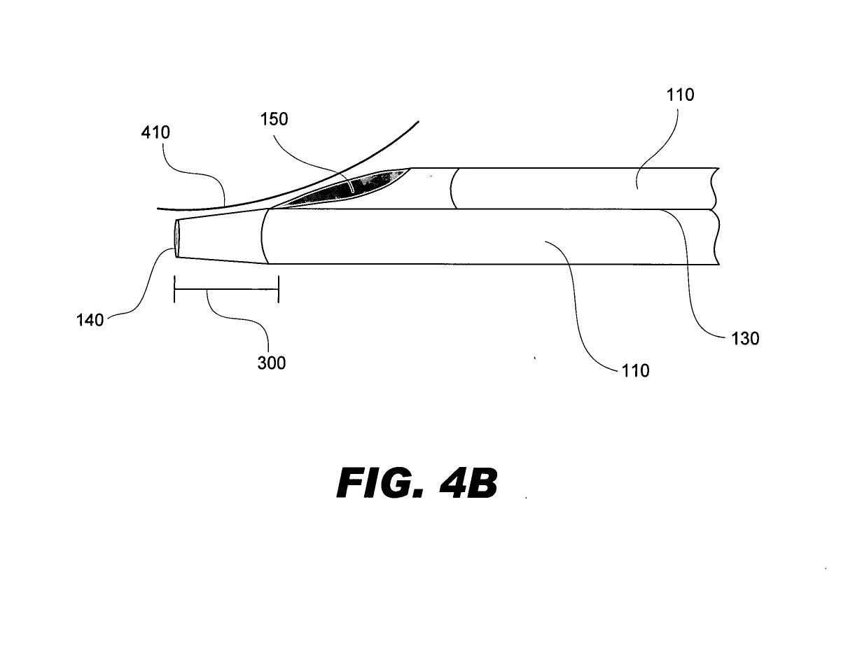

FIGS. 4A and 4B are schematic diagrams of the distal ports of a multiple lumen epidural introducer, according to various exemplary embodiments.

According to the embodiment of FIG. 4, a distal port (150) is steeply tapered to allow the epidural introducer (100) to enter a hole in the skin without catching on the tissue during entry or inhibiting internal passage. In some embodiments, multiple distal ports (140, 150) may be tapered together form a substantially smooth junction, such as a substantially consistent angle (400; FIG. 4A) or a curve (410; FIG. 4B).

As illustrated in FIG. 4A, a single angle (400) allows the multiple lumen epidural introducer to be inserted with minimal complications. A variety of angles (400) may be formed by the distal ports (140, 150) depending on the intended use of the epidural introducer (100). For example, an introducer to be inserted deep into a tissue may have a longer taper and a smaller corresponding angle (400).

As illustrated in FIG. 4B, the distal ports (140, 150) may form a curve (410). Distal ports (140, 150), lumens (110), and/or other elements of the epidural introducer may form a curve that is convex, concave, or that includes concave, convex, and/or flat regions.

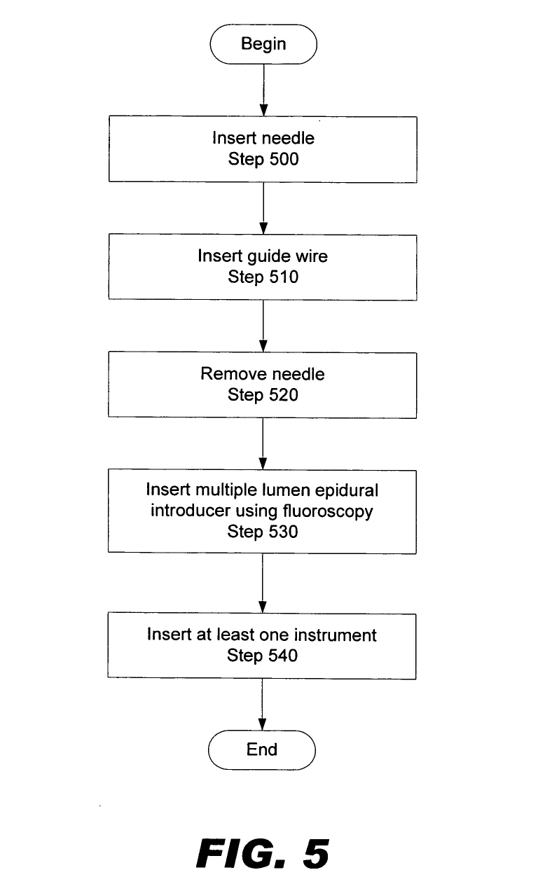

FIG. 5 is a flowchart illustrating a method of inserting and placing a multiple lumen epidural introducer, according to one exemplary embodiment.

First, a hollow needle is inserted into the spine to create a point of entry (step 500). The needle will pass through the skin of a patient and other tissue to create an entry site.

Next, a guide wire is inserted through the needle to maintain access to the entry site (step 510).

Once a guide wire is in place, the needle may be removed (step 520). The needle is carefully removed so as not to displace the guide wire.

A multiple lumen epidural introducer is then placed so as to gain access to the epidural space (step 530). The previously placed guide wire may be used to place the introducer. This is done, for example, by threading a proximal end of the guide wire into one of the lumens of the introducer and by then inserting the introducer along the guide wire into the epidural space. Additionally, fluoroscopic imaging may be used to place the introducer accurately and safely.

Once the introducer is in place, various instruments and devices may be inserted through the lumens of the introducer during the procedure (step 540). Devices may be inserted sequentially or simultaneously. The features of the multiple lumen epidural introducer, as described above, allow multiple devices to be inserted, manipulated, removed, and replaced through a single entry site.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A multiple lumen epidural introducer comprising:
at least two separate lumens each having a proximal port and a distal port; and
a coupling interface for coupling the two lumens such that the two separate lumens can be inserted through a single point of entry.

2. The introducer of claim 1, wherein said introducer is rigid enough for insertion through said point of entry to an epidural space of a patient.

3. The introducer of claim 1, wherein said coupling interface between said lumens is located at one or more of said distal ports.

4. The introducer of claim 1, wherein said coupling interface is configured to allow said lumens to be selectively uncoupled and re-coupled.

5. The introducer of claim 1, wherein a first said distal port is spaced from a second said distal port along a length of said introducer.

6. The introducer of claim 1, wherein at least a portion of said introducer is radio-opaque.

7. The introducer of claim 1, wherein at least one of said proximal ports comprises a luer lock.

8. The introducer of claim 1, wherein at least one of said distal ports comprises a tapered opening.

9. The introducer of claim 1, wherein at least one of said distal ports has an opening width less than an internal width of a corresponding lumen.

10. The introducer of claim 1, wherein at least one of said distal ports has an opening width greater than an internal width of a corresponding lumen.

11. The introducer of claim 1, wherein each of said lumens has a different internal diameter.

12. The introducer of claim 1, wherein said distal ports are formed to direct inserted instruments to sites separated by a first distance.

13. The introducer of claim 12, wherein said first distance is at least one millimeter.

14. The introducer of claim 1, wherein at least a portion of at least one of said lumens is flexible such that a distance between said proximal ports of said lumens can be selectively adjusted.

15. The introducer of claim 1, wherein said proximal ports of said lumens are spaced apart from each other.

16. The introducer of claim 1, wherein said lumens are isolated from each other such that there is no passage from an interior of a first of said lumens to an interior of a second of said lumens apart from said proximal and distal ports.

17. A method of using a multiple lumen epidural introducer comprising:
inserting two separate instruments respectively through two separate lumens, each lumen having a proximal port and a distal port;
wherein said two lumens are coupled at a coupling interface such that the two separate lumens can be inserted into a patient through a single point of entry.

18. A multiple lumen epidural introducer comprising:
means for providing at least two separate passageways for two respective instruments through patient tissue; and
means for coupling said two separate passageways such that said two separate passageways can occupy a single point of entry into said patient tissue.

19. The introducer of claim 18, wherein said means for coupling comprise means for releasably coupling said two passageways.

20. The introducer of claim 18, wherein said passageways are isolated from each other such that there is no passage from an interior of a first of said passageways to an interior of a second of said passageways apart from proximal and distal ports.
